(19) United States
(12) Patent Application Publication
(10) Pub. No.: US 2006/0063745 A1
(43) Pub. Date: Mar. 23, 2006

(54) Composition for relieving pain and improving mobility

(76) Inventor: Charles Berman, New York, NY (US)

Correspondence Address: Stephen E. Feldman, P.C., 12 East 41st Street, New York, NY, 10017, US

(21) Appl. No.: 10/947,510

(22) Filed: Sep. 22, 2004

(51) Int. Cl. A61K 31/65

(52) U.S. Cl. 514/154

(57) ABSTRACT

There is disclosed a composition for relieving pain and improving the mobility of a patient in need of such treatment. The composition comprises a tetrtacyclene compound and a non-steroidal compound. When administered concurrently, the composition compounds appear to have a potentiating effect. In one instance, the tetracyclene compound appears to potentiate the activity of the non-steroidal compound in relieving crippling pain and stiffness that impede mobility. In another Instance, the non-steroidal compound appears to potentiate the tetracyclene compound to down regulate inflammatory regulators.

### FIELD OF THE INVENTION

This invention is directed to a composition that relieves pain and, at the same time, improves mobility. More particularly, the composition of this invention comprises two separate and different compounds. Even more particularly, one of the compounds of the invention composition is tetracycline and the other is a non-steroidal compound.

### BACKGROUND OF THE INVENTION

Many people suffer from arthritis bursitis, neuralgia, myocitis, and the like which often cause inflammation and stiffness in their joints and muscles making it difficult, and painful to move their extremities. The joints and extremities affected the most include the shoulder, elbow, wrist, hip, knee, ankle and the knuckles of the hands and feet as well as the neck and back.

As disclosed in U.S. Pat. No. 6,063775 to Berman, tetracycline are known to be useful in the treatment of the HIV virus and AIDS.

Non-steroidal compounds are generally known to be useful to alleviate pain such as headache, backache, and the like.

However, there are no known medications or therapeutic agents that both alleviate pain and improve mobility of a person's extremities and back movement.

### SUMAMRY OF THE INVENTION

It has now been found that a person suffering from pain and impaired mobility can find significant additional relief from the composition of the present invention. In general, the composition of the present invention comprises a tetracycline compound often in sub-therapeutic doses, often in sub-antibiotic doses, and a non-steroidal compound.

Persons in need of being treated for both pain and impaired mobility are concurrently administered a therapeutically effective amount of a tetracycline compound and a therapeutically effective amount of a non-steroidal compound.

While the mechanism of action between the tetracycline compound and the non-steroidal compound is not completely understood, it appears that the presence of the tetracycline compound potentiates the activity of the non-steroidal compound in relieving the crippling pain and severe stiffness that impedes mobility, and or the NSAID potentiates the tetracycline to down regulate inflammatory mediators such as matrixmmp's.

The tetracycline compounds of the invention that can be used are members of the group consisting of 4-dimethylaminotetracyclene, 5a, 6-anhydro-4hydroxy-4-dedimethylaminotetracyclene, 4-dedimethylamino-11-hydroxy-12a-deoxytetracyclene, 12a-4a-anhydro-4-dedimethylaminotetracyclene, 6a benzylthiomethylaminotetracyclene. The 2-nitro analog of tetracycline(tetracyclonitrile), tetracycline pyrazola, 5a, 6-anhydro-4-hydroxy-4dedimethyltetracyclene, 7-chloro-4dedimethylaminotetracyclene, 4-dedimethylamino-7-chlorotetracycline, tetracycline altered at the 3 carbon position to produce a nitriletetracyclonitrile, 11-alpha-chloroyeyracyclene, 6-dimethyl-7-chloroyeyracyclene, 6-alpha-benzylthiomethylenetetracyclene, 11-alpha-chloroyeyracyclene, 6-dimethyl-6-deoxy-5-hydroxy-6-methylenetetracyclene 5a, 6-anhydro-4-hydroxy-4-dedimethylaminotetracyclene, 6a-deoxy-5-hydroxy-4-dedimethylaminotetracyclene, 7-chloro-6-dimethyl-4-dedimethylaminotetracyclene, 4-de(dimethylamino)-5-oxytetracycline, 7-chloro6-dimethyl-4dedimethylaminotetracyclene, 4-de(dimethylamino)-5-oxytetracycline, 6-alpha-deoxy-5-hydroxy-4-dedimethylaminotetracyclene, 7-chloro-6-dimethyl-4-dedimethylaminotetracyclene, 6-alpha-benzylthiomethylene-tetracycline, 6-fluoro-dimethyl-tetracycline their salts, conjugates and/or derivatives and combinations thereof

When tetracycline are used to treat the HIV virus or AIDS, typical dosage amounts range from about 0.1 mg/kg/day to about 50 mg/kg/day depending upon the particular tetracycline employed.

The non-steroidal compounds that can be used in the composition of the invention are members of the group consisting of Cox 2 and Cox 1 and 2 inhibitors of refecoxib, celeoxib, imcloxicam, naproxen, fenoprofen, melenamic acid, salsalate, indomethacin, ibuprofen, sulindac, disclofenic, piroxicam, naproxen, tolmetin, choline, oxaprozin, disclofenic, nabumetone, flubiprofin as well as combinations and mixtures thereof.

Depending upon the particular non-steroidal compound selected to treat pain, the dosage amounts used can vary from about 12.5 mg/day to about 50 mg/day to from about 100 mg. to about 300 mg. twice a day (BID) to about 50 mg. three times a day (TID) to from about 7.5 mg. To about 200 mg. four times a day (QID).

The tetrocyclenes can be used in sub-antibiotic doses.

What is claimed:

1. A composition for relieving pain and improving muscle movement and mobility comprising:
(a) a tetracycline compound; and
(b) a non-steroidal compound.

2. A method for relieving pain and improving mobility comprising:
concurrently administering to a patient in need of such treatment a therapeutically effective amount of a tetracycline compound and a therapeutically effective amount of a non-steroidal compound.
